(19) United States
(12) Patent Application Publication
(10) Pub. No.: US 2010/0144892 A1
(43) Pub. Date: Jun. 10, 2010

(54) METHOD OF PRODUCING ORGANIC CERTIFIED GLYCERIN

(75) Inventors: Jerry Wu, San Jose, CA (US); Brien Quirk, Eugene, OR (US); Tian X. Wang, San Jose, CA (US)

Correspondence Address: PROCOPIO, CORY, HARGREAVES & SAVITCH LLP, 530 B STREET, SUITE 2100, SAN DIEGO, CA, 92101, US

(73) Assignee: DRACO NATURAL PRODUCTS, INC., San Jose, CA (US)

(21) Appl. No.: 12/635,403

(22) Filed: Dec. 10, 2009

Related U.S. Application Data
(60) Provisional application No. 61/121,265, filed on Dec. 10, 2008.

(51) Int. Cl. A61K 31/045; C12P 7/20; A23L 1/00

(52) U.S. Cl. 514/738; 435/159; 426/60; 426/531

(57) ABSTRACT

A process for the production of naturally and organically fermented glycerin includes cleaning and sanitizing of the fermentation equipment prior to fermentation without using any traditional synthetic organic hydrocarbon-based chemicals. The fermentation process uses a non-chemical nutrient and nitrogen source from natural proteins during the fermentation to reduce trace amounts of toxins or contaminants. A purification stage following the fermentation process increases the purity of the glycerin to a desired level.

### RELATED APPLICATIONS

The present application claims the benefit of co-pending U.S. provisional patent application No. 61/121,265, filed on Dec. 10, 2008, the contents of which are incorporated herein by reference in their entirely.

### BACKGROUND

1. Field of the Invention

This invention relates to the production of organic glycerin or glycerol and to compositions containing organic glycerin, and is particularly concerned with methods for producing high purity organic glycerin which include cleaning and sanitizing steps for the equipment used in the method.

2. Related Art

Glycerin is a colorless, odorless, viscous liquid with a wide range of commercial and industrial applications. It is a major raw material in the manufacture of foams, including polyurethane foams. In the food and beverage industries, glycerin is often used as a solvent, sweetener and moisturizer in preparing “low-fat or low-glycemic foods.” In scientific research, glycerin is a common component of solvents. In medical and pharmaceutical and personal care preparations, glycerin is applicable as a base, lubricant and humectants. Glycerin is also found in cough syrups, elixirs and expectorants, toothpaste, mouthwashes, skin care products, shaving cream, hair care products, and soaps. It is a major raw material in the manufacture of foams, including polyurethane foams.

In recent years, a significant demand for highly refined glycerin has emerged. In particular, glycerin that equals or exceeds 97% in purity and meets the standards of US Pharmacopeia (USP) and FCC (Food Chemical Codex) has received particular attention for a wide range of applications in the global market.

Glycerin may be produced by the fermentation of a glucose source. One critical step in the process is in the pre-fermentation cleaning and sanitizing of the process equipment such as extraction tanks, boilers, filtering equipment and pipelines to remove dirt, contaminants and microorganisms before inoculating the appropriate microorganism to produce the desired products. In the post-fermentation phase this cleaning procedure is repeated to eliminate cross contaminations and to ensure that there is not a buildup of organic matter for microbial growth. In common practice, water and/or hot water and/or steam is/are used for pre-fermentation/post-fermentation cleaning and sanitizing, together with synthetic petro-chemical cleaners and disinfectants such as quaternary ammonium compounds, which are toxic to fish as effluents, or phenol based organic compounds. These synthetic chemical cleaners and disinfectants often contain trace substances such as dioxin, formaldehyde and other environmentally toxic, cancer-causing and immune suppressive agents.

While glycerin produced in a process employing synthetic chemicals is less expensive, it may retain chemical residues that are harmful and undesirable. For example, it is known that synthetic petro-chemicals often contain trace amounts of dioxin, methanol, formaldehyde, and other cancer-causing and immune suppressive agents.

Synthetically produced glycerin is not considered as a natural, sustainable, or organically certifiable ingredient for products that are consumed or applied to the human body, or sold in the organic market. As more and more consumers desire cosmetic, personal care, food, nutraceutical and pharmaceutical products derived from non-synthetic chemicals, a naturally and preferably an organically produced and organically certifiable glycerin is considered a desirable ingredient.

The fermentation process traditionally includes the use of urea, which is often provided as a nitrogen source to enhance the fermentation. Urea can react with ethanol to form ethyl urea, which is a know carcinogen compound. Also, substances such as diammonium phosphate and ammonium nitrate are disallowed by United States Department of Agriculture for “Natural Organic Products” and European Union Organic certification.

Accordingly, there is a need for a process for making organic glycerin using only ingredients allowed by organic certification bodies in the fermentation process, as well as a method of cleaning and sanitizing glycerin production equipment without the use of the traditional synthetic chemicals. It is also desirable that glycerin as an ingredient for use in food, cosmetic and pharmaceutical preparations should be free of any residue synthetic chemicals and/or toxic compounds.

### SUMMARY

According to one embodiment, a naturally and organically produced glycerin is produced by way of a fermentation process without the use of any synthetic, toxic petrochemicals or hydrocarbon-based chemicals. In preparation for the production, equipment used in the process, such as pipes, kettles, storage tanks, fermentation chambers, filters/filter media, and other devices are first cleaned and sanitized to remove dirt, contaminants, microorganisms and the like. This is undertaken by applying a cleaning medium, such as water, hot water, pressurized super-heated steam, hydrogen peroxide, potassium iodine, an acid solution, peroxyacetic acid, an alkaline solution, an inorganic halide compound, or the combination of any of them in the processing equipment. Free of synthetic petro-chemicals, any inorganic compounds used, which are ionic in nature, can be easily removed in the purification process.

According to another embodiment, a fermentation process for production of organic glycerin uses organic certified corn, yeast extract or other natural plant protein sources as the nutrient and nitrogen sources for the inoculation of the appropriate microorganism to sustain its growth as it produces glycerin.

Other features and advantages of the present invention will become more readily apparent to those of ordinary skill in the art after reviewing the following detailed description and accompanying drawings.

### BRIEF DESCRIPTION OF THE DRAWINGS

The details of the present invention may be gleaned in part by study of the accompanying drawings, in which like reference numerals refer to like parts, and in which:

FIG. 1 is a flow chart illustrating one embodiment of a method for making organic glycerin including an equipment cleaning process and a fermentation process for producing certified organic glycerin;

FIG. 2 is a more detailed flow chart illustrating the cleaning process;

FIG. 3 is a more detailed flow chart illustrating one embodiment of the fermentation process of FIG. 1; and

FIG. 4 illustrates one embodiment of a two stage distillation system used at one stage of the fermentation process of FIG. 3.

### DETAILED DESCRIPTION

Certain embodiments as disclosed herein provide for an oxygenated fermentation method of producing organic glycerin including steps of cleaning and sanitizing the process equipment prior to the fermentation steps using cleaning media which are easily removed and should not leave any toxic or synthetic chemical residue. The fermentation process uses organic certified corn or other natural plant protein sources as the nitrogen source, rather than urea, to enhance the fermentation. This meets natural and organic standards and allows refining of glycerin to a higher level of purity to meet standards for foods, cosmetics, and pharmaceutical compositions containing glycerin.

After reading this description it will become apparent to one skilled in the art how to implement the invention in various alternative embodiments and alternative applications. However, although various embodiments of the present invention will be described herein, it is understood that these embodiments are presented by way of example only, and not limitation. As such, this detailed description of various alternative embodiments should not be construed to limit the scope or breadth of the present invention.

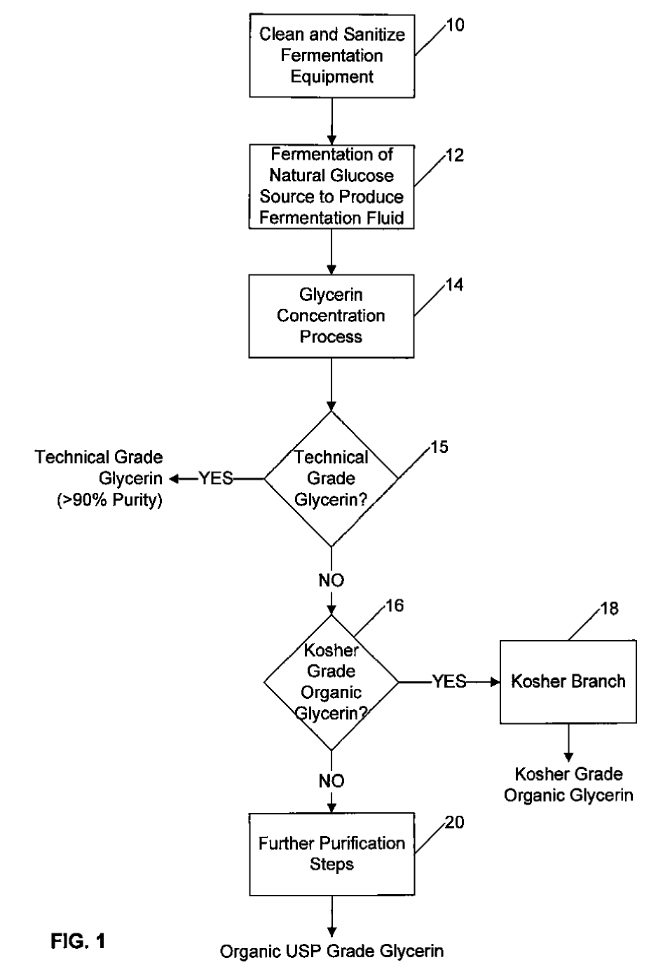

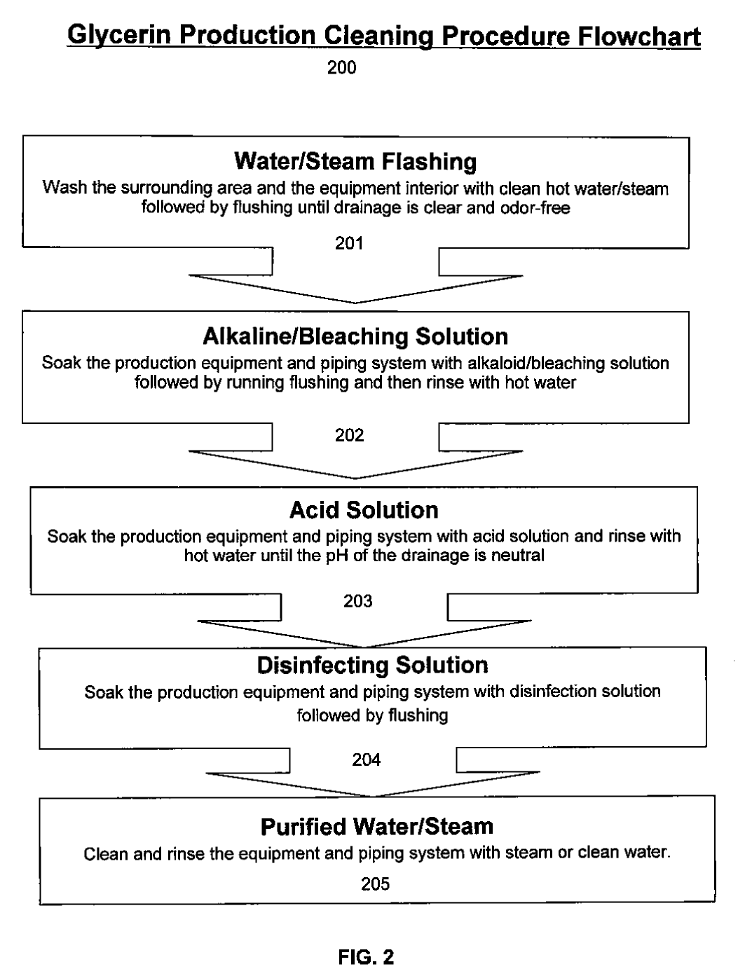

The basic steps of one embodiment of a process for preparation of organically certified glycerin are illustrated in FIG. 1. As illustrated, the fermentation equipment is first cleaned and sanitized (step 10). The cleaning and sanitizing step takes place prior to each fermentation procedure. In the cleaning and sanitizing step, the process equipment, such as pipes, kettles, storage tanks, bio-reactors, fermentation chambers, filters/filter media, concentrators, and other process equipments are first cleaned and sanitized to remove dirt, contaminants, microorganisms and the like. This procedure uses only inorganic chemicals or materials which are easily removable, and is described in more detail below in connection with FIG. 2. The cleaning medium or media used in this process may comprise water, hot water, steam, hydrogen peroxide, potassium iodine, an acid solution, an alkaline solution, an inorganic halide compound, or any combinations of any of the foregoing media.

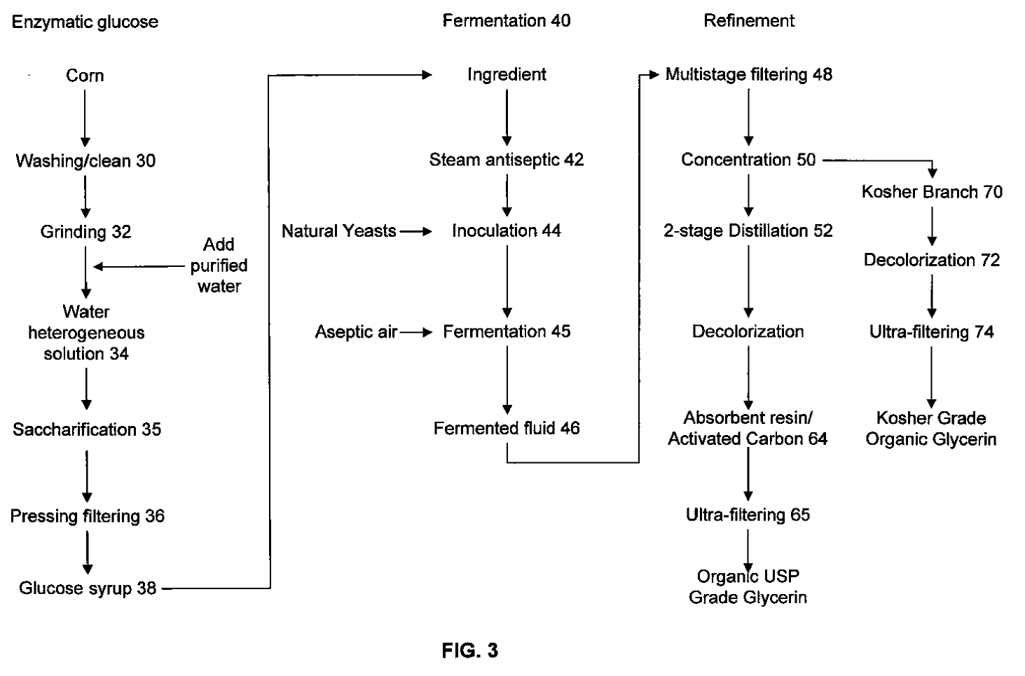

Following the cleaning and sanitization step, the fermentation process is carried out (step 12), as described in more detail below in connection with FIG. 3. The fermented fluid may be further refined or concentrated to achieve the desired purity level (step 14). In step 14, to achieve a higher purity level of, for example, 90% and above, which is considered a “Technical Grade” applicable to wide commercial and industrial applications, further concentration/purification can be achieved by vacuum evaporation, successive centrifugal and/or pressure filtering, distillation, and/or other processes as known in the art. For cosmetic and pharmaceutical applications, the liquid glycerin can also be filtered by using selectively permeable membrane and/or distillation to achieve a higher purity level, for example 99% or above, in compliance with the definition of the “US Pharmacopeia,” or “USP” Grade. In this case, other enzymes such as glucose oxidase with or without catalase are used to remove trace amounts of residual glucose which would otherwise cause yellowing of product and be caramelized during distillation. Alternatively, oxygenation by aeration followed by anaerobic conditions may be employed to ferment trace, remaining glucose to alcohol or additional glycerin, after which alcohol was readily evaporated from the medium. In step 15, if the purity level is sufficient, the process ends and technical grade glycerin is extracted from the equipment for subsequent use. If Kosher grade glycerin is desired (step 16), a Kosher branch (step 18) of the process (FIG. 3) involves additional processing steps to produce Kosher grade organic glycerin. If a higher purity level is required, further concentration steps are carried out (20), until a desired concentration level is reached. For example, for USP (United States Pharmacopeia) grade organic glycerin, the concentration level required is 99% or higher. Once the desired level is reached, USP grade organic glycerin is extracted from the equipment. The cleaning and sanitizing steps are then repeated prior to the next fermentation procedure.

As noted above, glycerin produced by conventional processes may contain chemical residues. These chemical residues are results of cleaning processes which deploy synthetic petro-chemical cleaners and disinfectants such as phenol-based or quaternary ammonium compounds. FIG. 2 illustrates one embodiment of an equipment cleaning procedure that can be used prior to a fermentation process in producing natural and organic glycerin which is substantially free of contaminants.

The cleaning process may use natural, non-toxic mineral type salts such as Sodium Hydroxide, Sodium Carbonate, Sodium Silicate, phosphate salts, and Polyphosphate salts. Sodium hydroxide is used to dissolve residual fats, grease, or oily compounds as residue on the stainless steel processing tanks by virtue of its saponifying capability (can convert fats into water soluble soaps). It also dissolves cell membranes of bacteria to destroy them in a caustic manner that is non-toxic after it is diluted and neutralized. Sodium Silicate is derived from sand and lye and it is used to protect stainless steel from corrosion. Phosphate salts and polyphosphate salts are also helpful in dissolving oils, fat and grease. They may be derived from a naturally mined phosphate mineral. This combination of ingredients works together to clean processing equipment without the use of petro-chemical or hydrocarbon derived agents like Sodium dodecyl benzene sulfonate, or quaternary ammonium compounds that are toxic to the environment and may contain dangerous trace contaminants like dioxin or other cancer-causing chemicals.

The cleaning process illustrated in FIG. 2 does not use any synthetic, toxic, petro-chemicals or hydrocarbon-based chemicals. In preparation for the production of organic glycerin, all equipment used in the process, such as pipes, kettles, storage tanks, bio-reactors, fermentation chambers, filters/filter media, concentrators, distillers, and other process equipment is first cleaned and sanitized to remove dirt, contaminants, microorganisms and the like. This is undertaken by applying a cleaning medium, such as water, hot water, pressurized superheated steam, hydrogen peroxide, potassium iodine, an acid solution, peroxyacetic acid, an alkaline solution, an inorganic halide compound, or the combinations of any of them in the processing equipment. Free of synthetic petro-chemicals, any inorganic compounds used, which are ionic in nature, can be easily removed in the purification process.

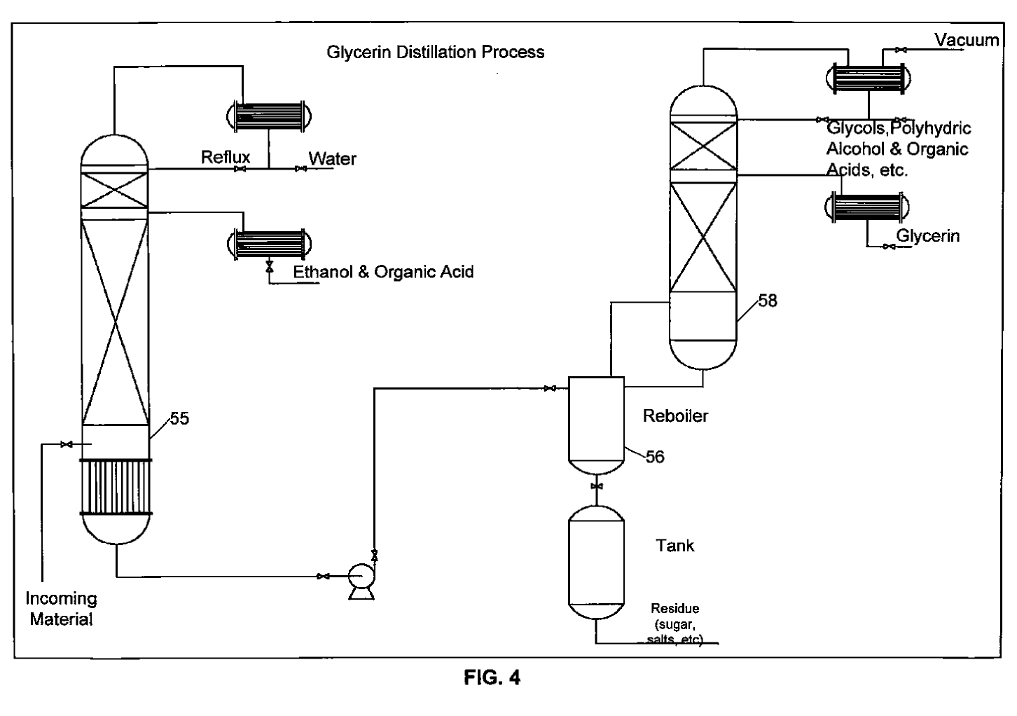

FIG. 2 illustrates one embodiment of an equipment cleaning and sanitizing process 200 that is performed prior to the glycerin production process of FIGS. 3 and 4. For purposes of illustration, the cleaning process 200 is divided into five major steps. The first step 201 comprises a water/steam flushing step in which the equipment is washed with clean hot water or steam and then flushed. The second step 202 comprises cleaning the equipment with an alkaline or bleaching solution. The third step 203 comprises cleaning with an acid solution. The fourth step 204 comprises cleaning with a disinfecting solution, while the fifth and final step 205 comprises cleaning with purified water or steam, as described in more detail below.

In step 201, the interior of the production equipment and piping system and the surrounding area are thoroughly washed with clean water. The equipment is then filled with clean water to a desired water level, and the water is circulated for a predetermined time period, such as ten minutes, in a cleaning circulation step. The circulation is stopped and the liquid is drained out of the equipment. The equipment interior is then washed with hot water (20-45° C.) and the water inside the equipment is completely drained. The equipment is then filled with hot water (40-80° C.) and/or steam, and pressurized circulation is run until the drainage is clear and odor-free.

Following the initial cleaning and sanitization, the apparatus is washed with a flushing solution, which comprises an alkaline or bleaching solution (block 202). This flushing solution further reduces the likelihood of contamination with materials from prior productions using the equipment. The flushing solution dissolves any protein or oil residues from previous productions, which might have oxidized and adhered to the interior of the production equipment. At block 202 (Cleaning with alkaline/beaching solution), the following steps are carried out:

1. Soak the production equipment and piping system with 0.05-5% alkaline solution. This is heated up to 60-80° C. for 1-2 hours
2. Start normal cleaning circulation for 10-20 minutes
3. Stop the circulation and drain the solution inside the equipment completely
4. Wash the equipment interior with hot water (40-60° C.) and then fill the equipment with clean water to the corresponding water level
5. Run forced circulation for 10-20 minutes and then drain the water inside the equipment

In the above step, an alkaline solution of sodium/calcium/potassium hydroxide may be used with one or a mixture of sodium carbonate, sodium silicate, phosphate salt, and polyphosphate salt. The alkaline solution may comprise an aqueous solution of sodium hydroxide, calcium hydroxide, potassium hydroxide, sodium carbonate, sodium silicate, phosphate salt, and polyphosphate salt. The amount of the alkaline solution may be applied at a concentration of 0.05% by weight or above. In one example, an aqueous solution of sodium hydroxide (NaOH), sodium hypochlorite (NaOCl) or a combination of NaOH and NaOCl (for example 5% (w/w) solution) is used as the flushing solution. In another example, a 1% w/w solution of sodium hydroxide in deionized filtered water may be used as a flushing solution to wash and dissolve residues of materials from prior production operation. In another example, an aqueous solution of 5% w/w of a hypochlorite salt is used to sanitize and wash away undesirable microbes from the production equipment.

At block 203 (Cleaning with acid solution), the following steps are carried out:

1. Soak the production equipment and piping system with 0.05-5% acid solution for 0.5-2 hours.
2. Start the cleaning circulation for 10-20 minutes
3. Stop the circulation and drain the solution inside the equipment
4. Wash the equipment interior with hot water (40-60° C.)
5. Run forced circulation for 10-20 minutes and then drain the water inside the equipment
6. Open the lids of the equipment and rinse the equipment interior until the pH of the drainage is neutral by litmus paper

In the above steps, the acid solution may be water and one or more of phosphoric acid, citric acid, lactic acid, peracetic acid, peroxyacetic acid (derived from acetic acid which is found in vinegar and hydrogen peroxide, two simple, non-persistent household cleaning agents), sulphur dioxide, hydrogen peroxide, calcium citrate, and ascorbic acid (vitamin C).

At block 204 (Cleaning with disinfecting solution), the following steps are carried out:

1. Soak equipment with 0.01-2% detergent/disinfecting solution for 15 minutes.
2. Start the cleaning circulation for 5 minutes.
3. Stop the circulation and drain the sanitation (disinfecting) solution inside the equipment.

The disinfecting solution used at block 204 may be water and an inorganic halide or oxyhalide. The inorganic halide or oxyhalide may be a chlorine bleach solution, which comprises one or more of sodium hypochlorite, calcium hypochlorite, calcium chloride, potassium hypochlorite, potassium chloride, chlorine dioxide, sodium and chlorine dioxide solution as chlorite solution. The amount of the chlorine bleach solution may be applied at a concentration of 0.05% by weight or above. Alternatively, the disinfecting solution may be water and one or more of hydrogen peroxide, potassium iodide, phosphoric acid, citric acid, lactic acid, peracetic acid, peroxyacetic acid, calcium citrate, and sulfur dioxide.

Following the preceding flushing steps with alkaline solution (202), acid solution (203) and disinfecting solution (204), all parts of the production equipment, including the fermentation apparatus, are again flushed with a water or steam wash to rinse away any possible traces of inorganic compounds, which might remain in the production equipment. Generally, the cleaning process is considered adequate when the total inorganic compounds in the outlet are 0.01% or less as determined by the water-loss method or mineral ash. At block 205 (Cleaning with purified water and steam), the following steps are carried out:

1. Clean and rinse the equipment and pipeline system one more time with steam or purified water
2. Record all cleaning actions taken, and display Equipment Status Notice

In the event that water, alkaline solutions, acid solution, and/or chlorine bleach solutions are applied without steam in any of the foregoing steps, it is preferable that they are introduced at a temperature of at least 10 degree C.

The aforementioned cleaning and sanitization steps are undertaken before the fermentation run for the production of glycerin is undertaken. For repeated fermentation runs thereafter, the application of steam may be applied as necessary to maintain the cleanliness and sanitation of the production equipment.

FIG. 3 is a flow chart illustrating one embodiment of a detailed process for oxygenated fermentation of glycerin. Following the cleaning and sanitization steps of FIG. 2, a glucose source derived from an organic certified starch-containing product, such as organically certified corn flour, is introduced into the fermentation apparatus to inoculate the appropriate microorganism to produce the fermented glycerin. Alternatively, another organically certified enzymatic glucose source, such as the starch from corn, potato, rice, sweet potato or sorghum, can be substituted. Where the glucose source is corn, quality assurance is carried out to verify that the starting raw material is certified organic corn, by verification of USDA National Organic program (NOP) and/or European Union (EU/EEC), vendor certified lot paperwork, and quality assurance testing for specifications (starch content, aflatoxins, water level, etc.).

At step 30, inspection and cleaning of the corn is carried out, specifically: visual inspection of corns; and cleaning with water to remove residual cob pieces, dust, chaff and foreign materials. This is followed by steeping and grinding, which may include the following steps: Place the corns in 50-60° C. water for 20-40 hrs in a stainless steel steeping tank. Corns absorb water during this time, resulting in an increase in their moisture level from about 15% to 45%. After steeping, corns are coarsely ground to break the germ loose from other components. Steeping water is condensed to capture nutrients in the water for use in animal feeds and as a yeast starter nutrient for later fermentation processes. The ground corn in water slurry flows to the next stage.

Germ Separation: a physical separator spins the low density corn germs out of the corn and water slurry. The germ, containing approximately 85% of the oil present in the corn, is pumped onto screens and washed repeatedly to remove any starch left in the mixture. (A series of mechanical processes extracts the oil from the germs. The oil can then be refined and filtered into finished corn oil.) The high protein germ residue is saved as another useful component of animal feeds.

Fine grinding and filtering (32): The corn and water slurry leaves the germ separator and goes through a more thorough grinding in an impact or attrition-impact mill to release the starch and gluten from the fiber. The suspension of starch, gluten and fiber flows over fine screens which catch fiber but allow starch and gluten to pass through. The fiber is piped to the feed house for drying and for later use as a major ingredient of animal feeds. The starch-gluten suspension, or mill starch, is piped to the next step.

Starch separation: Gluten has a low density compared to starch. By passing mill starch through a centrifuge, the gluten is readily spun out for use in animal feeds. The starch, now diluted, is washed an additional 8 to 14 times in hydroclones to remove the last trace of protein to yield high quality starch.

Liquefier or Syrup conversion (34): Starch water solution, 25-40% starch suspended in water, is liquefied in the presence of amylase that converts the starch to a low-dextrose solution. First, the starch water solution is mixed with amylase and heated at 102-110° C. for 3-10 minutes, then kept in the solution liquefier at 85-95° C. for 60-120 minutes.

Saccharification (35): Liquefied starch is mixed with glucoamylase, a natural amylase, and maintained in the saccharification vessel at the temperature of 50-65° C. for 60-120 minutes. During the saccharification, the solution in the vessel is stirred at a speed of 50-100 rpms. The solution is then filtered (36) to produce glucose syrup (38).

Sterilization (42): The glucose syrup, which contains 25-40% glucose, is sterilized by ultra-filtration (0.2-0.05 μm) and/or steam to kill microbes that may be present.

The fermentation procedure (40) is then carried out. During fermentation, a seeding material is seeded in the fermentation apparatus to effect the fermentation of the glucose syrup. The seeding material may be a natural yeast. The fermentation of the glucose syrup results in a fermentation fluid containing glycerin, which is then filtered to separate the glycerin liquid from the fermentation residue, as discussed in more detail below. The filtering can be by means of membrane filter or other suitable means. Further concentration steps may be carried out in order to achieve a desired higher purity level.

One embodiment of the fermentation procedure, as illustrated in FIG. 3, comprises the following steps:

Inoculation (44): The sterile syrup from step 42 is the substrate in the fermentation process which is first inoculated by the addition of a seeding material comprising a natural yeast such as Candida Krusei, a naturally derived yeast that has specific glycerin-formation capabilities.
Fermentation (45): Since the glycerin formation process has a high demand for oxygen, an optimized aerobic fermentation process is performed with filtered aseptic air pumped into the fermentation chamber. The fermentation procedure 45 in one embodiment is a two-phase process designed to obtain a higher glycerin production rate with less than 0.5% residual glucose. Phase 1 of the two-phase process produces glycerin from glucose syrup by oxygenated fermentation. The oxygenated fermentation is controlled at temperature of 28-39° C. and stirring speed of 90-200 rpm for 60-120 hours. To optimize the fermentation, aseptic air is pumped into the bioreactor at a specific rate of 0.5 to 1.0 M³/1000 L (V/V) per minute. The pumping in of air is stopped when glucose in the fermentation fluid is converted to less than 4%.

The fermentation then continues to the second phase, which comprises anaerobic fermentation. In the anaerobic fermentation phase, the air in the bioreactor is pumped out, creating a vacuum to reduce oxygen levels. The process runs at a temperature range of 30-38° C. for 24-48 hours. The second phase of the fermentation is designed to significantly reduce glucose, a difficult residue to be removed in the purification process, to a level of less than 0.5%.

An alternative procedure for the second phase of the fermentation process is to add special bacteria, Corynebacterium, into the bioreactor, and then to run the process at a temperature range of 32-38° C. for 6 to 12 hours. The level of residual glucose can then be reduced to less than 0.5%.

The fermentation fluid 46 produced from the two phase fermentation procedure described above is then refined. The first step of refinement is a multistage filtering procedure (48) in which fat, yeast putty, protein, polysaccharides, sugar, and other impurities in the fermented fluid are separated and removed as solids by an ultra-filtration system. For further refining, glycerin liquid may be filtered by a nano-filtration system with a MWCO 100, and/or other processes as known in the art. The filtrate should be perfectly transparent prior to diverting it to the concentration stage 50. The solid phase is collected for other uses with further processing through a pressure filter. The liquid phase or filtrate is diverted to additional purification steps in the concentration stage 50 for production of the desired grade of organic glycerin.

In one embodiment of concentration stage 50, glycerin liquid produced by the multistage filtering 48 is concentrated by using a reverse osmosis (RO) system and/or vacuum concentrator to remove excess water. The concentration process involves RO technology removing water by using membrane dialysis and/or heating the hot liquid glycerin under pressure and subsequently spraying the glycerin into a vacuum chamber where water evaporates off as steam and glycerin remains a liquid.

Following the concentration stage 50, a two stage distillation 52 is carried out. FIG. 4 illustrates one embodiment of a two stage distillation system which may be employed to distill glycerin liquid to obtain a minimum 99% of purity. In the first stage of distillation, the glycerin liquid is heated to 100-135° C. in a first distillation column 55 to remove water, alcohol, and organic acid. The concentrated glycerin is heated by a high-efficiency reboiler 56 to a temperature of 140-180° C. The vapor of the glycerin fluid is driven to a second distillation column 58 under a vacuum less than 60 mmHg, where glycerin is separated from other residues, such as organic acids, glucose, polyhydric alcohols, etc. The distillation process also reduces the color and odor of glycerin fluid (62). The 2-stage distillation system in this invention is designed to achieve the following goals, 1) to obtain a high purity of 99% glycerin, 2) to avoid glycerin decomposition, which starts at 190° C., and 3) to maintain energy efficiency.

To obtain USP grade glycerin, the distilled glycerin is first polished by ultra-filtration using absorbent resin and/or fine activated carbon treatment 64 (FIG. 3) to remove impurities and coloring agents. Efficient treatment with food-grade absorbent resin and/or fine activated carbon may be achieved by repeating the resin or carbon “bleaching” for substantially impurities in the glycerin.

Next, ultra-filtration or additional filtration 65 is performed to eliminate substantially all remaining impurities that may be present. At the end of this step, the quality of glycerin meets USP standards, and has excellent color stability upon heating.

In some cases, the process may be modified to produce Kosher grade glycerin rather than USP grade glycerin. Kosher grade organic glycerin preserves more water from the natural fermentation process than USP grade glycerin.

The process of producing Kosher grade glycerin is illustrated in the process branch labeled as KOSHER BRANCH 70 in FIG. 3. In the Kosher branch, distillation, resin and evaporation steps are modified or avoided to preserve more naturally occurring water content in the final glycerin product. The Kosher branch includes decolorization step 72 and ultra-filtering step 74 to produce Kosher grade glycerin containing 50%-70% glycerin, as compared to USP grade glycerin which contains more than 97% glycerin.

To meet natural and organic standards, no synthetic organic carbon compound (i.e. hydrocarbons, urea) is added during the fermentation process for producing USP grade or Kosher grade organic glycerin. The foregoing process uses botanic sources that have high level of protein and/or whey protein as nutrients. Plants with high protein also provide the nitrogen source needed during fermentation, so that use of urea as a nitrogen source can be avoided. In the foregoing process of glycerin fermentation, plant protein may be added to the corn starch, with the ratio of corn starch to plant protein and/or whey protein and/or ammonium carbonate or ammonium bicarbonate being in the range from 100:0.25 to 100:8.25. Examples of possible botanic sources for use as nutrients in the foregoing fermentation process are listed below.

One possible nutrient source is plants with high protein including Quinoa (Chenopodium quinoa), Rice (Oryza sativa), Sorghum (Sorghum bicolor), Corn (Zea mays) and Soy (Glycine max).

Another good protein source is amaranth (Amaranthus caudatus), which is easily harvested, and produces a lot of fruits (and thus seeds) which are used as grain. It contains large amounts of protein and essential amino acids, such as lysine. Due to its weedy life history, amaranth grains grow very rapidly and their large seedheads can weigh up to 1 kilogram and contain a half-million seeds. Amaranthus species are reported to have 30% higher protein value than other cereals, such as rice, wheat flour, oats and rye.

Pulses are another good source of protein. Pulses are 20 to 25% protein by weight, which is double the protein content of wheat and three times that of rice. For this reason, pulses are sometimes called “poor man's meat”. While pulses are generally high in protein, and the digestibility of that protein is also high, they often are relatively poor in the essential amino acid methionine. Grains (which are themselves deficient in lysine) are commonly consumed along with pulses to form a complete protein diet. The Food and Agriculture Organization of the United Nations (FAO) recognizes 11 primary pulses:

1. Dry beans (Phaseolus spp. including several species now in genus Vigna):
a. Kidney bean, haricot bean, pinto bean, navy bean (Phaseolus vulgaris)
b. Lima bean, butter bean (Vigna lunatus)
c. Azuki bean, adzuki bean (Vigna angularis)
d. Mung bean, golden gram, green gram (Vigna radiata)
e. Black gram, Urad (Vigna mungo)
f. Scarlet runner bean (Phaseolus coccineus)
g. Rice bean (Vigna umbellata)
h. Moth bean (Vigna acontifolia)
i. Tepary bean (Phaseolus acutifolius)
2. Dry broad beans (Vicia faba):
a. Horse bean (Vicia faba equina)
b. Broad bean (Vicia faba)
c. Field bean (Vicia faba)
3. Dry peas (Pisum spp.):
a. Garden pea (Pisum sativum var. sativum)
b. Protein pea (Pisum sativun var. arvense)
4. Chickpea, Garbanzo, Bengal gram (Cicer arietinum)
5. Dry cowpea, Black-eyed pea, blackeye bean (Vigna unguiculata)
6. Pigeon pea, Toor, cajan pea, congo bean (Cajanus cajan)
7. Lentil (Lens culinaris)
8. Bambara groundnut, earth pea (Vigna subterranea)
9. Vetch, common vetch (Vicia sativa)

### 10. Lupins (Lupines spp.)

11. Minor pulses, including:
a. Lablab, hyacinth bean (Lablab purpureus)
b. Jack bean (Canavalia ensiformis), sword bean (Canavalia gladiata)
c. Winged bean (Psophocarpus teragonolobus)
d. Velvet bean, cowitch (Mucuna pruriens var. utilis)
e. Yam bean (Pachyrrizus erosus)

At certain stages in the glycerin fermentation process described above, pH value may need to be adjusted by adding either acid or alkaline (base). The pH value may be adjusted by use of acid, minerals, and vitamin sources. The stages where adjustment of the pH value is needed in the process include:

A “Double Enzyme Method” is used for starch conversion to glucose. Amylase is used in the first stage to convert large starch polymers into smaller oligomeric dextrins. In the second stage, glucoamylase converts dextrins to glucose. Amylase and glucoamylase have different optimal pH, and may require acid or alkaline addition to adjust pH. Starch's natural pH is about 4.5, and in order to meet the high temperature tolerant enzyme, a-amylase's optimum pH is 5.8-6.5, so alkaline compounds must first be added to starch to adjust the pH.

After liquefiction, acid must be added to adjust pH value to 4.0 to 5.5, which is the optimal pH for glucoamylase. Acid and alkaline addition is required to adjust pH to optimal value.

At the inoculation and seeding stage, pH value is controlled at 6.0-7.5.

Fermentation medium and fermentation process control: pH value must be monitored and maintained at a pH value level of 3.0-4.5 by acid and alkaline solution addition.

Post-Fermentation (before evaporation), acidic fermentation broth must be neutralized to pH value to 6.5-8.0.

The pH value and other conditions in the saccharification process stage may be controlled as follows:

Liquefiction: The pH range is from 5.0-8.0, and the temperature is in the range from 85-110° C.

Saccharification: Glucoamylase, also known as glycoamylase, scientific name: α-1,4-glucose hydrolase (α-1,4-Glucan glucohydrolase), pH range is about 3.0-4.5, and the temperature range is about 50-65° C.

The pH value and other conditions in the fermentation process stage may be controlled as follows:
1. Inoculation and seeding stage: The pH value ranges from 6.0-7.5, the stirring speed is from 90 to 200 rpm, and the temperature is 28 to 39° C.
2. Fermentation: The pH range is about 3.0 to 4.5, the stirring speed is from 90 to 200 rpm, and the temperature range is 28 to 39° C.

Acids, including one or more of citric acid, lactic acid, acetic acid and phosphoric acid, and/or sodium hydroxide, and/or calcium hydroxide, and/or potassium hydroxide, are used for the purpose of adjusting pH value.

Moreover, in the fermentation process, plant protein, trace minerals, nutrient minerals, and nutrient vitamins are added in fermentation mediums. For the plant proteins, the range of usage is 0.05% to 5% by weight. For the rest, the range of usage is 30 ppm to 1%. Nutrient vitamins include ascorbic acid (Vitamin C), Vitamin Bs, Vitamin E, and the like.

N may also be added with nutrient minerals P, K, Ca, Mg, etc.:

K (potassium chloride, potassium iodide, potassium carbonate, potassium phosphate, and/or potassium hydroxide),

P (sodium phosphates, and/or potassium phosphate),

Mg (magnesium sulfate, magnesium carbonate, magnesium chloride, and/or magnesium stearate) and

Ca (calcium chloride, calcium citrate, calcium chloride, and/or calcium hydroxide).

Increasingly, more consumers want cosmetic, personal care, food, and pharmaceutical preparations based on natural products, not those that are made from chemical or synthetic products. Fermentation glycerin, in particular organic certified glycerin, is most suitable for such products. In addition, most cosmetic, personal care, food, and pharmaceutical products contain anti-oxidants. Anti-oxidants are not stable in such products. They can be oxidized and lose their activity and performance. The glycerin fermentation process uses special sources of starch, such as corn starch, to produce glycerin containing trace amounts of vitamins which support healthy cell growth or anti-oxidant status. These vitamins, such vitamin C an E, the carotenoids, such as lutein and zeaxantin, and naturally occurring phenolic compounds can help prevent the oxidization of anti-oxidants.

The glycerin production process described above employs corn or other plant or natural protein sources as the nitrogen source instead of urea for the inoculation of the appropriate microorganism to sustain its growth as it produces glycerin. The glycerin is refined to a higher degree of purity to meet the standards for organic cosmetics, food, and pharmaceutical applications. Derived from a naturally produced glucose source, the fermented glycerin produced in the above process is free of synthetic organic chemicals commonly encountered in the art. Glycerin derived from non-organic feedstock contains traces of pesticide contaminants. The process described above, using organic or naturally produced feedstock, provides for a fermentation-produced glycerin which can safely support a wide range of applications such as in the production of food, cosmetic and pharmaceutical products.

Organic glycerin produced in the above fermentation process contains small amount of vitamins and trace metals, such as iron, magnesium, zinc, copper, chromium, manganese, vanadium, molybdenum and selenium, which are considered desirable for the support of human health and well being and nourishment for the skin. Where desirable, additional vitamins and/or trace minerals may be added to the glycerin when used as a composition for a cosmetic preparation.

Glycerin is used to accelerate wound healing for skin cells. With higher levels of antioxidants and vitamins naturally contained in Kosher certified glycerin, and a greater level of water for hydrating, this composition will have a greater effect at promoting wound healing. Trace elements zinc and copper contained in corn derived glycerin are needed for collagen production and skin cell proliferation.

The fermentation process described above for producing Kosher or higher (USP) grade glycerin meets the standards and requirements of USDA National Organic program (NOP) and/or of the European Union (EU/EEC), and the federal regulations of organic production. The glycerin obtained from the fermentation process may be used in organic certified products of cosmetic, food, dietary supplement or pharmaceutical applications.

Glycerin produced by the foregoing processes has also been observed to contain naturally occurring trace amounts of organic acids, such as citric acid, acetic acid, lactic acid, pyruvic acid, malic acid, and ketoglutaric acid, which are considered desirable for the support of human skin health and well being and nourishment for the skin and hair

Examples of natural products manufactured using organic glycerin produced by the above embodiments are described below. The glycerin listed in each of the following examples may be a selected grade level of glycerin manufactured according to any of the processes described above. It should be noted that when Kosher grade organic glycerin is used in these products, the added water portion in the formula would be proportionately reduced, as Kosher grade glycerin contains 30%-50% of water.

### Example 1

Ingredients or formulation for a natural production foundation which complies with the requirements for an ECOCERT organic cosmetic product are listed in the Table 1.

TABLE 1
Organic Liquid Foundation

| Seq | INCI Name | % |
|---|---|---|
| 1 | Water | QS |
| 2 | Sucrose | 1 |
| 2 | Sucrose Stearate | 1.5 |
| 2 | Caffeine | 0.1 |
| 2 | Magnesium Aluminum | 0.4 |
| | Silicate | |
| 2 | Hyaluronic | 0.02 |
| | Acid | |
| 3 | Glycerin | 10 |
| 3 | Xanthan Gum | 0.2 |
| 4 | Iron Oxide | 0.88 |
| 4 | Iron Oxide | 0.336 |
| 4 | Titanium Oxide | 5.28 |
| 4 | Iron Oxide | 0.072 |
| 4 | Lecithin | 1 |
| 4 | Mica | 0.5 |
| 5 | Simmondsia Chinensis | 5 |
| | (Jojoba) Seed Oil | |
| 5 | Camellia Sinensis Leaf Oil | 0.25 |
| 5 | Helianthus Annuus | 5.25 |
| | (Sunflower) Seed Oil | |
| 5 | Oryza Sativa (Rice) Bran Wax | 0.5 |
| 5 | Astrocaryum Murumuru Seed | 0.1 |
| | Butter | |
| 5 | Butyrospermum Parkii (Shea | 8 |
| | Butter) | |
| 5 | Glyceryl stearate | 1.5 |
| 5 | Theobroma Cacao (Cocoa) | 0.5 |
| | Seed Butter | |
| 5 | Cetyl Alcohol | 0.5 |
| 5 | Sorbitan Sesquioleate | 0.3 |
| 6 | glucose oxidase, | 0.001 |
| | lactoperoxidase | |

A procedure to produce a natural product foundation using the ingredients of Table 1 comprises:

1. Weigh sequence (seq) 1 and heat to 85° C.
2. Sprinkle in seq 2 ingredients while mixing fast and mix for 30 mins.
3. Premix then add seq 3 to seq 1, 2 under a high shear mixer such as those manufactured by Silverson Machines or an Epinbach homomixer BEFORE emulsifying.
4. Premix seq. 4 and grind the pigments in a high shear mixer for 20 minutes. Then add mixture to seq. 1-3.
5. Premix seq. 5 ingredients at 85° C., then add to seq 1-4 in a high shear mixer or Silverson mixer for five to ten minutes.
6. Cool to 40° C. then add seq. 6.

### Example 2

Ingredients of body wash for complying with requirements of ECOCERT organic cosmetic product are listed in the Table 2.

TABLE 2
Ingredients of Natural Body Wash

| Seq | INCI Name | % |
|---|---|---|
| 1 | Water | QS |
| 1 | Tetrasodium EDTA | 0.2 |
| 1 | Citric Acid | 0.15 |
| 1 | Glycerin | 10 |
| 1 | Sorbitol | 1 |
| 2 | Aloe Barbadensis Leaf Juice | 0.02 |
| 2 | Polyquaternium-10 | 0.05 |
| 3 | Sodium Coco Sulfate | 4 |
| 3 | Peg-150 Pentaerythrityl tetras | 1 |
| 3 | Sodium Lauryl Sarcosinate | 0.5 |
| 3 | Sodium Cocoyl Sarcosinate | 3 |
| 3 | Peg 80 Sorbitan Laurete | 0.05 |
| 3 | Cocamidopropyl Betaine | 6 |
| 4 | Sodium Chloride | 0.5 |
| 5 | glucose oxidase, lactoperoxidase | 0.001 |

One procedure to produce natural body wash using the foregoing ingredients comprises:

1. Weigh seq. 1 and set under prop—no heat.
2. Sprinkle in seq. 2 and mix thoroughly 30 mins.
3. Now heat to 80° C. Once materials reach 80° C. add in seq. 3 ingredients one at a time in the order listed.
4. Add seq. 4 once mixture reaches 40 to 50° C. Continue mixing.
5. Add seq. 5 while mixing, discharge at 32° C.

One skilled in the field may modify the foregoing natural body wash formula to develop other types of cleansing products, such as shampoo, facial cleanser, and the like for the face, body, hair, and hands, by using different types of viscosity modifiers and actives.

### Example 3

Ingredients of natural mascara are listed in the Table 3.

TABLE 3
Ingredients of Natural Mascara

| Seq | INCI Name | % |
|---|---|---|
| 1 | Water | QS |
| 1 | Glycerin | 5 |
| 1 | Simethicone | 0.05 |
| 1 | Potassium Hydroxide | 4 |
| 1 | Maltodextrin | 4 |
| 2 | Beeswax | 13 |
| 2 | Stearic Acid | 7 |
| 2 | Simmondsia Chinensis (Jojoba) Seed Oil | 1 |
| 3 | Iron Oxide | 2.84 |
| 3 | Lecithin | 0.5 |
| 3 | Water | 15 |
| 4 | Glucose and Glucose Oxidase and | 0.001 |
| | Lactoperoxidase | |

One embodiment of a procedure to produce natural mascara using the ingredients in Table 3 comprises:

1. Add Seq 1 ingredients while mixing until uniform.
2. Pre-mix seq. 2 ingredients at 85° C.
3. Pre-mix Seq 3 and grind the ingredients with Silverson high shear mixer for ten minutes.
4. Add Seq 3 to Seq 1 under prop: then heat to 80° C.
5. Heat Seq 2 to 85° C. using Silverson for ten minutes.
6. Air cool batch to 40° C. under slow paddle, and add seq. 4.
7. Drop batch at 30° C.

### Example 4

A formula of ingredients for a natural lipstick is listed in the Table 4:

TABLE 4
Ingredients of Natural Lipstick

| Seq | INCI Name | % |
|---|---|---|
| 1 | Beeswax | 2.25 |
| 1 | Euphorbia Cerifera (Candelilla) Wax | 14.4 |
| 1 | Copernicia Cerifera (Carnauba) Wax | 0.8 |
| 1 | Theobroma Cacao (Cocoa) Seed Butter | 4.7875 |
| 1 | Ricinus Communis (Castor) Seed Oil | 27.0625 |
| 1 | Simmondsia Chinensis (Jojoba) Seed Oil | 4.25 |
| 1 | Butyrospermum Parkii (Shea Butter) | 3.5 |
| 1 | Phenoxyethanol | 0.2 |
| 1 | Glycerin | 3 |
| 2 | Lecithin | 1 |
| 2 | Titanium Dioxide | 4.1 |
| 2 | D&C Red 6 Barium | 0.2 |
| | Lake | |
| 2 | D&C Red 7 | 0.5 |
| | Calcium Lake | |
| 2 | Ricinus Communis (Castor) Seed Oil | 36.95 |

A procedure to produce natural lipstick using the foregoing components comprises:

1. Premix and grind seq. 2.

2. Heat seq. 1 to 80° C. and add premixed seq. 2 with mixing.

3. Pour mixture to component.

### Example 5

Ingredients of natural face lotion are listed in table 5.

TABLE 5
Ingredients of natural face lotion

| Seq | INCI Name | % |
|---|---|---|
| 1 | Water | 55.78 |
| 1 | Glycerin | 10 |
| 1 | Sucrose | 0.5 |
| 1 | Sucrose Stearate | 2 |
| 2 | Hyaluronic Acid | 0.01 |
| 3 | Camellia Sinensis Leaf Oil | 2 |
| 3 | Helianthus Annuus (Sunflower) Seed Oil | 5 |
| 3 | Oenothera Biennis (Evening Primrose) Oil | 0.05 |
| 3 | Glyceryl Stearate | 1 |
| 3 | Steareth-20 | 0.9 |
| 3 | Sorbitan Sesquioleate | 0.2 |
| 3 | Oryza Sativa (Rice) Bran Wax | 1 |
| 3 | Simmondsia Chinensis (Jojoba) Seed Oil | 3 |
| 3 | Astrocaryum Murumuru Seed Butter | 0.01 |
| 3 | Butyrospermum Parkii (Shea Butter) | 5 |
| 3 | Theobroma Cacao (Cocoa) Seed Butter | 1 |
| 3 | Cetearyl Alcohol | 2 |
| 4 | ORGANI ALCOHOL | 10 |

One example of a procedure of producing natural face lotion using the ingredients of Table 5 is:

### 1. Weigh seq 1

2. Sprinkle in seq 2 while mixing fast and mix for 30 mins.
3. Now heat to 75° C. Cover with foil to keep from evaporating.
4. Pre-mix seq 3 at 80° C. until uniform.
5. Add seq 3 to seq 1, 2 using Silverson or Epinbach high shear mixer for 10 minutes.
6. Cool the batch to 35° C. and add seq 4 with mixing.
7. Discharge batch at 32° C.

One skilled in the field may modify the natural lotion formula and develop other types of skin and hair care products, such as cream, serum, tonic spray, conditioner, etc. for the face, body, hair, and hands by using different types of viscosity modifiers and actives.

### Example 6

Ingredients of natural itch spray are listed in table 6.

TABLE 6
Ingredients of Natural Itch Spray

| Ingredients | % |
|---|---|
| Sophora flavescens extract | 0.35 |
| Licorice extract | 0.25 |
| Alcohol | 12 |
| Glycerin, | 30 |
| Slippery Elm | 0.30 |
| Chamomile extract | 0.10 |
| Water Purified | 51 |
| Vitamin C | 5% |
| Pantothenic acid | 1% |

Additional, mixed organic glycerin applications in foods, drugs, and cosmetics products are listed in following examples.

### Example 7

Pharmaceutical suppository laxative preparation containing at least 70% by weight of organically certified ingredients:

80.7 wt % organically certified glycerine USP grade,

11.2 wt % purified water,

8.1 wt % stearic acid.

### Example 8

OTC cough syrup containing at least 70% by weight organic produced ingredients:

22 wt % organic glycerin, USP,

33 wt % organic glucose,

17 wt % organic corn syrup, stevia,

28 wt %: Sodium Benzoate, Water Citric Acid, Beet Color, Natural Flavor, Guaifenesin USP (USP 100 mg), Dextromethorphan Hydrobromide (USP 10 mg) per 5 ml serving.

### Example 9

Gluten-free low-fat Ginger Apple Cinnamon soft cookies food product with made with at least 95% by weight organic produced ingredients:

35 wt % organically certified rice flour,

20 wt % organically certified sorghum flour,

10 wt % organically certified apple sauce,

10 wt % organically certified cane sugar,

5 wt % organically certified butter,

5 wt % tapioca starch,

6 wt % organically certified glycerin,

4 wt % organically certified black-strap molasses,

2 wt % organically certified ginger powder,

1 wt % cinnamon powder,

1 wt % baking powder,

1 wt % salt.

### Example 10

Pasteurized Lemon Iced Tea (Green Tea) drink made with low sugar/carbs, and containing 100% organic ingredients:

75 wt % filtered, deionized water,

5 wt % organically certified fresh squeezed lemon juice,

10 wt % brewed organically certified green tea leaves,

5 wt % organically certified green tea extract,

5 wt % organically certified glycerin.

### Example 11

“Organic” Isotonic Hydrating Work-out Drink made with at least 95% by weight organic produced ingredients:

Filtered water, 89.5 wt %,

5 wt % organic glycerin,

4 wt % organic cane sugar,

0.25 wt % salt,

0.4 wt % organic strawberry flavor,

0.15 wt % citric acid,

0.1 wt % sodium citrate,

0.1 wt % sodium benzoate.

### Example 12

“100% Organic” Liquid Pomegranate Whole Fruit extract with 0.7% ellagic acid in glycerin base. Sweet-tasting easily dispensed standardized botanical extract in a convenient dropper bottle for through-out the day use.

70 wt % organic pomegranate extract (containing 10% ellagic acid),

20 wt % organic glycerin,

10 wt % filtered, pasteurized water.

Following are more examples of personal care products.

### Example 13

“100% Organic” certified moisturizing lotion with 100% organic botanicals and lipids containing:

25 wt % organically certified glycerin,

30 wt % organically certified sunflower seed (Helianthus annua) lipids,

2 wt % organically certified hydrosol of Chamomile 1.2% flavonoids extract in 98% water (Chamomilla recutita), 3 wt %, 2 wt % organically certified hydrosol of Ural Licorice 12% Glycyrrhizin extract in 98% water (Chamomilla recutita),

5 wt %, organically certified lecithin,

2 wt %, organically certified rose water,

32 wt % water,

0.25 wt % organically certified rosemary oil.

### Example 14

Hair conditioner with 100% organic botanicals, humectants and lipids:

45.5 wt % purified water,

30 wt % organically certified glycerin,

6 wt % organically certified almond (Prunus amygdalus) lipids,

4 wt % organic kelp extract,

5 wt % organically certified hydrosol of rose petal,

5 wt % of lavender flower,

2 wt %, organically certified lecithin,

2 wt %, 0.5 wt % natural organic vitamin E from organic soybeans.

### Example 15

Shampoo containing at least 70% organic produced ingredients:

70 wt % deionized water,

15 wt % organic glycerin,

6 wt % coconut derived surfactants,

4 wt % organic slippery elm extract,

3 wt % organic kelp,

1 wt % organic yucca extract,

1 wt % organic jojoba oil.

### Example 16

Organic Lip Balm with 100% organic ingredients:

30% wt organic beeswax,

25 wt % organic glycerin,

20 wt % organic green tea seed oil,

11 wt % purified, water,

5 wt % organic sunflower seed oil,

3 wt % chamomile extract,

3 wt % organic honey,

2 wt % organic Goji berry extract,

1 wt % rosemary extract (std to 10% carnosic acid).

Increasingly, more consumers want cosmetic, personal care, food, and pharmaceutical preparations based on natural products, not those that are made from chemical or synthetic materials. The foregoing examples of products and many other natural products for human or animal use can be made using organic certified glycerin made according to the foregoing processes. The fermentation process uses only organic certified glucose sources derived from organic certified starch-containing substrates such as corn, sorghum, rice and the like. This is combined with the foregoing method of cleaning and sanitizing glycerin production equipment without the use of the traditional synthetic chemicals so as to substantially reduce or eliminate any residue synthetic chemicals or toxic compounds. The organically certified glycerin manufactured as described above also contains trace amounts of vitamins and minerals which are considered desirable for human and animal health as well as skin nourishment when used in a cosmetic or skin treatment product.

The above description of the disclosed embodiments is provided to enable any person skilled in the art to make or use the invention. Various modifications to these embodiments will be readily apparent to those skilled in the art, and the generic principles described herein can be applied to other embodiments without departing from the spirit or scope of the invention. Thus, it is to be understood that the description and drawings presented herein represent a presently preferred embodiment of the invention and are therefore representative of the subject matter which is broadly contemplated by the present invention. It is further understood that the scope of the present invention fully encompasses other embodiments that may become obvious to those skilled in the art and that the scope of the present invention is accordingly limited by nothing other than the appended claims.

1. A method of making organic certified grade glycerin, comprising:
cleaning all parts of a fermentation apparatus in a plurality of successive cleaning steps in which a cleaning solution is circulated through the fermentation apparatus repeatedly for a predetermined time period;
the cleaning steps comprising at least circulation of water or steam through the apparatus, and circulation of at least one of an alkaline solution, an acidic solution, and sodium hypochlorite through the apparatus;
introducing a natural glucose source into the fermentation apparatus;
hydrolyzing the glucose source to produce sugar;
filtering the hydrolyzed glucose source to produce glucose syrup;
fermenting the glucose syrup with a seeding material comprising a selected natural yeast to produce a glycerin/syrup mixture;
filtering the glycerin/syrup mixture to separate glycerin liquid from syrup residue; and
further concentrating the separated glycerin liquid to produce a purity level corresponding to a selected grade of organic glycerin.

2. The method of claim 1, wherein the cleaning steps further comprise a final cleaning step of washing out the fermentation apparatus with water or steam until inorganic compounds detected in the outlet are no more than 0.01% by weight;

2. The method of claim 1, wherein said alkaline solution consists of at least one of the following bases or salts: sodium hydroxide, calcium hydroxide, potassium hydroxide, sodium carbonate, sodium bicarbonate, sodium silicate, phosphate salt, magnesium sulfate, and polyphosphate salt.

3. The method of claim 1, wherein said acid solution consists of at least one of the following acids: phosphoric acid, citric acid, lactic acid, alginic acid, peracetic acid, peroxyacetic acid, sulphur dioxide, sodium citrate, and acetic acid.

4. The method of claim 1, wherein said cleaning solutions each have a temperature of at least 10° C. during circulation.

5. The method of claim 1, wherein said cleaning steps further comprise circulation of a disinfecting solution through the apparatus after circulation of at least one of the acidic and alkaline solution.

6. The method of claim 5, wherein said disinfecting solution comprises an inorganic halide solution.

7. The method of claim 6, wherein the inorganic halide consists of at least one of sodium hypochlorite, hydrogen peroxide, calcium hypochlorite, calcium chloride, calcium citrate, calcium hydroxide, monobasic calcium phosphates, potassium hypochlorite, potassium chloride, potassium iodide, chlorine dioxide, sulfur dioxide, sodium phosphates, and chlorine dioxide solution.

8. The method of claim 6, wherein the alkaline solution, acid solution, and inorganic halide compound solution each have a concentration of at least 0.05% by weight.

9. The method of claim 1, wherein the natural yeast used in the fermentation process is Candida krusei yeast.

10. The method of claim 1, wherein the natural glucose source comprises a glucose source which is organically certified by the United States National Organic Program (NOP) or by the European Union (EU/ECC).

11. The method of claim 10, wherein the glucose source comprises a high protein botanic source.

12. The method of claim 10, wherein the glucose source is a starch selected from the group consisting of corn, potato, rice, sweet potato, and grain sorghum.

13. The method of claim 1, wherein the glucose source contains trace amounts of vitamins, nutrient minerals, and organic acids, whereby the glycerin produced by the fermentation also contains trace amounts of vitamins, minerals and organic acids.

14. The method of claim 1, further comprising using a feed stock which is selected from the group consisting of vegetable source protein, whey protein, ammonium carbonate, and ammonium bicarbonate.

15. The method of claim 14 wherein said vegetable source protein is rice, wheat, soy, corn, sorghum, quinoa, amaranth, grain, or pulses as defined by the Food and Agriculture Organization of the United Nations (FAO).

16. The method of claim 1, wherein the hydrolysis step comprises enzymatic lipase hydrolysis.

17. The method of claim 1, wherein the fermenting step is a two phase fermentation process comprising a first phase of oxygenated fermentation.

18. The method of claim 17, wherein the two phase fermentation process further comprises a second phase of anaerobic fermentation.

19. The method of claim 17, further comprising a second fermentation phase which comprises adding Corynebacterium to the fermentation fluid when the level of glucose in the fermentation fluid is detected to be less than 4% and continuing the fermentation until the level of glucose is reduced to less than 0.5%.

20. The method of claim 1, comprising continuing the fermentation until the level of glucose in the fermentation fluid is less than 0.5%.

21. The method of claim 1, wherein the glucose source is organically certified, the level of glucose in the separated glycerin produced by the fermentation process is no more than 0.5% and the glycerin is organically certifiable by at least one organic certification body as an organic glycerin product.

22. The method of claim 21, further comprising mixing a predetermined amount of the purified organic glycerin with additional compounds in a predetermined formulation to produce an organic cosmetic, food, dietary supplement or pharmaceutical composition.

23. A method for the production of organic glycerin by fermentation comprising:
a first fermentation phase comprising oxygenated fermentation of a fermentation medium comprising glucose syrup and a naturally occurring yeast in a bioreactor at a temperature of 28-40° C. with a stirring speed of 90-200 rpm for 80-120 hours; and
a second fermentation phase comprising anaerobic fermentation of the fermentation product of the first phase to reduce glucose, the second fermentation phase comprising pumping out air from the bioreactor is pumped out to produce a vacuum and running the fermentation process at a temperature range of 30-38° C. for 24-48 hours to produce a post fermentation broth.

24. The method of claim 23, wherein the yeast is a Candida krusei yeast.

25. The method of claim 23, further comprising adding acid or alkaline solution to the fermentation medium during the oxygenated fermentation stage to maintain the pH value of the fermentation medium in the range from 3.0 to 4.0.

26. The method of claim 25, further comprising adding the yeast to the glucose syrup at an inoculation and seeding stage prior to the first fermentation phase, and controlling the pH of the fermentation medium during the inoculation and seeding stage to be in the range from 6.0 to 7.5.

27. The method of claim 25, further comprising neutralizing the post fermentation broth to a pH value between 6.5 to 8.0.

28. A method for the production of organic glycerin, comprising:
conducting a first fermentation phase comprising oxygenated fermentation of glucose syrup with a naturally occurring yeast in a bioreactor at a temperature of 28-40° C. with a stirring speed of 90-200 rpm for 80-120 hours;
monitoring the level of glucose in the fermentation fluid;
when the level of glucose in the fermentation fluid is less than 4% by weight, adding Corynebacterium into the bioreactor and continuing the fermentation at a temperature range of 32 to 38° C. for six to twelve hours until the level of residual glucose is reduced to less than 0.5% by weight.

29. The method of claim 28, further comprising controlling the pH value to be in a predetermined range during each fermentation stage by adding acid or alkaline solution.

30. A method for the production of organic glycerin, comprising:
fermenting a glucose syrup produced from a natural glucose source to produce a glycerin liquid containing a trace amount of glucose;
heating the glycerin liquid to 100-135° C. in a first distillation column to remove water, alcohol, and organic acids and produce concentrated glycerin; and
heating the concentrated glycerin in a reboiler to a temperature of 140 to 180° C. and directing the resultant vapor of the glycerin fluid into a second distillation column under a vacuum less than 60 mmHg to further concentrate the glycerin.

31. A method for the production of organically certifiable glycerin, comprising:
cleaning all parts of a fermentation apparatus in a plurality of successive cleaning steps including at least one step of circulation of water or steam through the apparatus, the cleaning steps using no synthetic hydrocarbon based chemicals;
producing a glucose syrup from a naturally occurring protein source;
fermenting the glucose syrup with a seeding material comprising Candida krusei yeast to produce a glycerin/syrup mixture;
filtering the glycerin/syrup mixture to separate glycerin liquid from syrup residue; and
further concentrating the separated glycerin liquid to produce a purity level corresponding to a selected grade of organic glycerin.

32. An organic cosmetic, food, dietary supplement or pharmaceutical composition, comprising a mixture of compounds including glycerin in the range from 3% to 81% by weight, the glycerin being certified as organic by an organic certification organization.

33. The composition of claim 31, wherein the glycerin contains trace amounts of vitamins, minerals and organic acids.
